(19) United States
(12) Patent Application Publication
(10) Pub. No.: US 2026/0011012 A1
(43) Pub. Date: Jan. 8, 2026

(54) AUXILIARY INFORMATION GENERATION METHOD, AUXILIARY INFORMATION GENERATION DEVICE, AND RECORDING MEDIUM

(71) Applicant: OLYMPUS MEDICAL SYSTEMS CORP., Tokyo (JP)

(72) Inventor: Shunsuke OKUMURA, Machida-shi (JP)

(73) Assignee: OLYMPUS MEDICAL SYSTEMS CORP., Tokyo (JP)

(21) Appl. No.: 19/335,740

(22) Filed: Sep. 22, 2025

Related U.S. Application Data
(63) Continuation of application No. PCT/JP2023/025083, filed on Jul. 6, 2023.

(51) Int. Cl. G06T 7/00; A61B 1/00; A61B 1/05; G06T 7/11; G06V 10/44; G16H 30/40

(52) U.S. Cl. CPC G06T 7/0012; A61B 1/00009; A61B 1/05; G06T 7/11; G06V 10/44; G16H 30/40; G06T 2207/30092

(57) ABSTRACT

An auxiliary information generation method includes sequentially acquiring a plurality of images obtained by an image sensor of an endoscope at a plurality of positions in a digestive tract of a subject, detecting feature regions in the digestive tract based on the plurality of images, calculating the number of the feature regions in each of a plurality of sections in the digestive tract, generating auxiliary information according to the number of the feature regions, and controlling a state of the auxiliary information according to the number of the feature regions when the auxiliary information is displayed on a display.

This application is a continuation application based on International Patent Application PCT/JP2023/25083 filed on Jul. 6, 2023, the contents of which are incorporated herein by reference.

### BACKGROUND OF THE INVENTION

### Field of the Invention

The present invention relates to an auxiliary information generation method, an auxiliary information generation device, and a recording medium.

### Description of Related Art

In recent years, a method of determining feature parts in an image by using image recognition technology and displaying the image and a result of the determination has been used in various scenes. However, when types or the number of detected feature parts increase, a display screen becomes filled with information on the result of the determination, which decreases visibility of the image.

Japanese Unexamined Patent Application, First Publication No. 2010-238098 discloses a technology for superimposing a graphic object on an image of real space. In this technology, a plurality of pieces of augmented reality information that are displayed close to each other are displayed as one aggregate graphic object.

### SUMMARY OF THE INVENTION

According to a first aspect of the present invention, an auxiliary information generation method includes: sequentially acquiring a plurality of images obtained by an image sensor of an endoscope at a plurality of positions in a digestive tract of a subject; detecting feature regions in the digestive tract based on the plurality of images; calculating the number of the feature regions in each of a plurality of sections in the digestive tract; generating auxiliary information according to the number of the feature regions; determining whether the image sensor is executing imaging in a depth direction of the digestive tract; and controlling a state of the auxiliary information according to the number of the feature regions when it is determined that the image sensor is executing imaging in the depth direction and the auxiliary information is displayed on a display.

According to a second aspect of the present invention, in the first aspect, the auxiliary information generation method may further include generating the auxiliary information for each of a plurality of image regions obtained by dividing an image obtained by the image sensor in a direction corresponding to a circumferential direction of the digestive tract.

According to a third aspect of the present invention, in the second aspect, the auxiliary information generation method may further include controlling a size of an icon indicating the auxiliary information for each of the plurality of image regions according to the number of feature regions.

According to a fourth aspect of the present invention, in the first aspect, the auxiliary information generation method may further include: dividing an image obtained by the image sensor in a direction corresponding to a radial direction of the digestive tract; and generating the auxiliary information for each of the plurality of image regions obtained by dividing the image in a direction corresponding to a circumferential direction of the digestive tract.

According to a fifth aspect of the present invention, in the first aspect, the auxiliary information generation method may further include: generating the auxiliary information displayed in a first state when the number of the feature regions is greater than or equal to a predetermined number; and generating the auxiliary information displayed in a second state different from the first state when the number of the feature regions is less than the predetermined number.

According to a sixth aspect of the present invention, in the fifth aspect, the auxiliary information displayed in the second state may indicate positions of the feature regions.

According to a seventh aspect of the present invention, an auxiliary information generation device includes a processor. The processor detects feature regions at respective positions along a lumen within the lumen based on an image obtained by an image sensor at a distal end of an endoscope to calculate the number of feature regions at the respective positions. The processor determines an imaging direction of the image sensor within the lumen. The processor generates auxiliary information according to the number of feature regions at the respective positions when the imaging direction is determined to be a depth direction of the lumen.

According to an eighth aspect of the present invention, a computer-readable non-transitory storage medium is provided, which has recorded a program causing a computer to execute: detecting feature regions at respective positions along a lumen within the lumen based on an image obtained by an image sensor at a distal end of an endoscope to calculate the number of feature regions at the respective positions; determining an imaging direction of the image sensor within the lumen; and generating auxiliary information according to the number of feature regions at the respective positions when the imaging direction is determined to be a depth direction of the lumen.

### BRIEF DESCRIPTION OF THE DRAWINGS

FIG. 1 is a block diagram showing an example of the configuration of an endoscope system according to a first embodiment of the present invention.

FIG. 2 is a diagram showing an example of a plurality of sections in the first embodiment of the present invention.

FIG. 3A is a diagram showing an example of an imaging direction in the first embodiment of the present invention.

FIG. 3B is a diagram showing an example of the imaging direction in the first embodiment of the present invention.

FIG. 4 is a flowchart showing an example of a procedure of processing executed by an auxiliary information generation device according to the first embodiment of the present invention.

FIG. 5 is a diagram showing an example of a method for calculating the number of unobserved regions in the first embodiment of the present invention.

FIG. 6 is a flowchart showing an example of a procedure of processing executed by the auxiliary information generation device according to the first embodiment of the present invention.

FIG. 7A is a diagram showing an example of an unobserved region detected in an image in the first embodiment of the present invention.

FIG. 7B is a diagram showing an example of an image and auxiliary information displayed on a display unit in the first embodiment of the present invention.

FIG. 8 is a flowchart showing an example of a procedure of processing executed by the auxiliary information generation device according to the first embodiment of the present invention.

FIG. 9A is a diagram showing an example of the unobserved region detected in the image in the first embodiment of the present invention.

FIG. 9B is a diagram showing an example of the image and the auxiliary information displayed on the display unit in the first embodiment of the present invention.

FIG. 10 is a flowchart showing an example of a procedure of processing executed by the auxiliary information generation device according to the first embodiment of the present invention.

FIG. 11 is a diagram showing an example of a display screen of the display unit in the first embodiment of the present invention.

FIG. 12 is a flowchart showing an example of a procedure of processing executed by an auxiliary information generation device according to a second embodiment of the present invention.

FIG. 13A is a diagram showing a first example of a display screen of a display unit in the second embodiment of the present invention.

FIG. 13B is a diagram showing a second example of the display screen of the display unit in the second embodiment of the present invention.

FIG. 13C is a diagram showing a third example of the display screen of the display unit in the second embodiment of the present invention.

FIG. 14A is a diagram showing a fourth example of the display screen of the display unit in the second embodiment of the present invention.

FIG. 14B is a diagram showing a fifth example of the display screen of the display unit in the second embodiment of the present invention.

FIG. 14C is a diagram showing a sixth example of the display screen of the display unit in the second embodiment of the present invention.

FIG. 15 is a flowchart showing an example of a procedure of processing executed by the auxiliary information generation device according to the second embodiment of the present invention.

FIG. 16 is a flowchart showing an example of a procedure of processing executed by the auxiliary information generation device according to the second embodiment of the present invention.

### DETAILED DESCRIPTION OF THE INVENTION

Hereinafter, an embodiment of the present invention will be described with reference to the drawings. An example of an endoscope system including an auxiliary information generation device will be described below. In the following description, “a plurality of XX” means two or more XX.

### First Embodiment

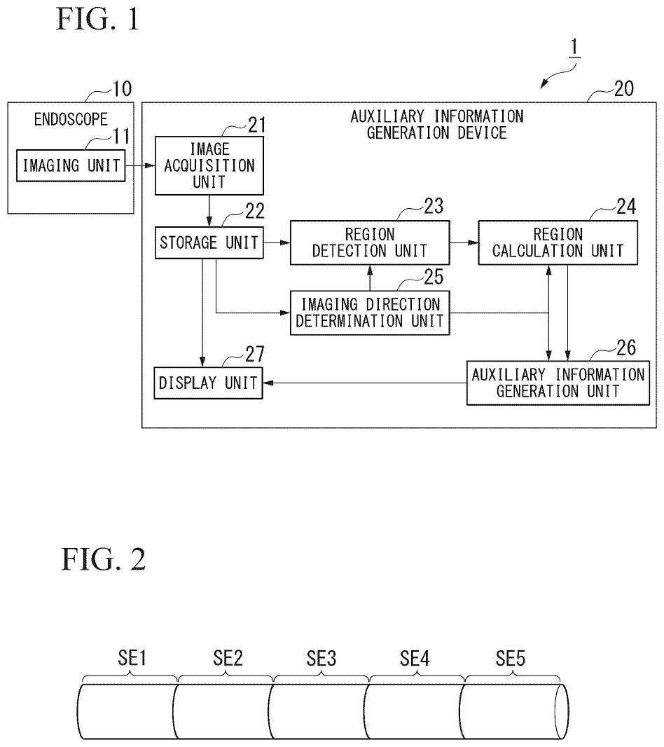

FIG. 1 shows an example of the configuration of an endoscope system 1 according to a first embodiment of the present invention. The endoscope system 1 shown in FIG. 1 has an endoscope 10 and an auxiliary information generation device 20.

The endoscope 10 is inserted into the digestive tract of a subject. For example, the digestive tract is a large intestine. The endoscope 10 has an imaging unit 11. The imaging unit 11 is disposed at the distal end of the endoscope 10. The imaging unit 11 is an image sensor, and generates a plurality of images at a plurality of positions within the digestive tract of the subject. The imaging unit 11 outputs the plurality of images to the auxiliary information generation device 20.

The auxiliary information generation device 20 has an image acquisition unit 21, a storage unit 22, a region detection unit 23, a region calculation unit 24, an imaging direction determination unit 25, an auxiliary information generation unit 26, and a display unit 27.

The image acquisition unit 21 sequentially acquires a plurality of images output from the imaging unit 11. The image acquisition unit 21 outputs the acquired plurality of images to the storage unit 22.

The storage unit 22 is a memory, and stores a plurality of images output from the image acquisition unit 21. The plurality of images stored in the storage unit 22 are sequentially output to the region detection unit 23, the imaging direction determination unit 25, and the display unit 27.

The region detection unit 23 detects feature regions in each of a plurality of sections in the digestive tract based on the plurality of images output from the storage unit 22. In the following example, an example in which the region detection unit 23 detects an unobserved region as a feature region will be described. There are cases where a region in the digestive tract may not be captured in the image, or visibility of the region in the image may be poor. Alternatively, there are cases where a region in the digestive tract may be captured in the image, but a doctor does not perform detailed observation or treatment of the region. The region detection unit 23 detects these regions as unobserved regions. The region detection unit 23 outputs region position information indicating the positions of the unobserved regions in the image to the region calculation unit 24.

FIG. 2 shows an example of the plurality of sections. The digestive tract is divided into sections SE1 to SE5. For example, each section is set based on a time when an image of a region in the section is captured, and includes regions whose images are captured within a predetermined length of time. Each section may have a predetermined length and include regions within that length. Each section may be set for each specific portion in the digestive tract.

The region calculation unit 24 calculates the number of unobserved regions in each of the plurality of sections in the digestive tract. The region calculation unit 24 outputs region number information indicating the number of unobserved regions in each section to the auxiliary information generation unit 26.

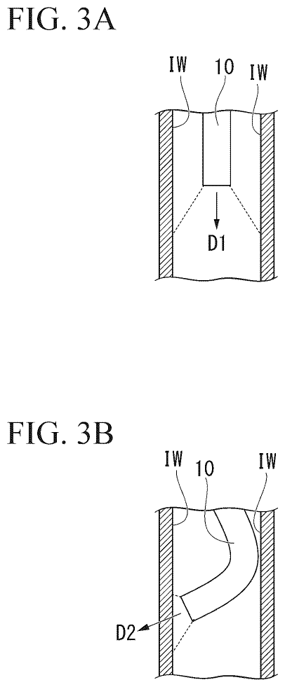

The imaging direction determination unit 25 determines an imaging direction of the imaging unit 11 based on the plurality of images output from the storage unit 22. FIGS. 3A and 3B show examples of the imaging direction.

In FIG. 3A, the imaging unit 11 executes imaging in a depth direction D1 (longitudinal direction) along an inner wall IW of the digestive tract. In FIG. 3B, the imaging unit 11 executes imaging in a direction D2 toward the inner wall IW. The doctor bends the endoscope 10 to perform observation or treatment on a specific region. At this time, the imaging unit 11 executes imaging in the direction D2. The imaging direction determination unit 25 may determine the imaging direction based on the state of a bending mechanism for bending the endoscope 10. The imaging direction determination unit 25 outputs imaging direction information indicating the imaging direction to the region calculation unit 24 and the auxiliary information generation unit 26.

When the plurality of images have changed and an image feature of a dark lumen hole is continuously detected near the center of the images, the imaging direction determination unit 25 may determine that the imaging direction is the direction D1. Alternatively, when the feature gradually disappears and a specific image feature is detected on a wall of a uniform lumen, the imaging direction determination unit 25 may determine that the imaging direction has switched to the direction D2. The imaging direction determination unit 25 may also determine the direction D1 and the direction D2 by using information detected by a special sensor or a sensor of an operation unit.

In addition, the imaging direction determination unit 25 determines a traveling direction of the endoscope 10 based on the plurality of images output from the storage unit 22. When the doctor moves the endoscope 10 toward an examination object in the digestive tract, the traveling direction of the endoscope 10 is the depth direction. When the doctor moves the endoscope 10 in a direction in which it is removed from the digestive tract, the traveling direction of the endoscope 10 is a backward direction. The imaging direction determination unit 25 outputs traveling direction information indicating the traveling direction of the endoscope 10 to the region detection unit 23.

The auxiliary information generation unit 26 generates auxiliary information according to the region number information output from the region calculation unit 24, and outputs the auxiliary information to the display unit 27. The auxiliary information is displayed in a state according to the number of unobserved regions. The auxiliary information generation unit 26 controls a state of the auxiliary information.

The display unit 27 is a liquid crystal monitor or the like. The display unit 27 sequentially displays a plurality of images output from the storage unit 22. Furthermore, the display unit 27 displays the auxiliary information output from the auxiliary information generation unit 26 together with the images.

At least one of the image acquisition unit 21, the region detection unit 23, the region calculation unit 24, the imaging direction determination unit 25, and the auxiliary information generation unit 26 may be constituted by a processor such as a central processing unit (CPU).

A computer may read a program and execute the read program. The program includes instructions that define an operation of at least one of the image acquisition unit 21, the region detection unit 23, the region calculation unit 24, the imaging direction determination unit 25, and the auxiliary information generation unit 26. That is, the functions of at least one of the image acquisition unit 21, the region detection unit 23, the region calculation unit 24, the imaging direction determination unit 25, and the auxiliary information generation unit 26 may be realized by software.

The program described above may be provided by a “computer-readable recording medium” such as a flash memory. The program may be transmitted from a computer that holds the program to the endoscope system 1 via a transmission medium or by a transmission wave in the transmission medium. The “transmission medium” that transmits the program is a medium that has a function of transmitting information. The medium that has the function of transmitting information includes a network (communication network) such as the Internet and a communication line (communication line) such as a telephone line. The program described above may realize a part of the functions described above. Furthermore, the program described above may be a differential file (differential program). The functions described above may be realized by a combination of a program already recorded in the computer and a differential program.

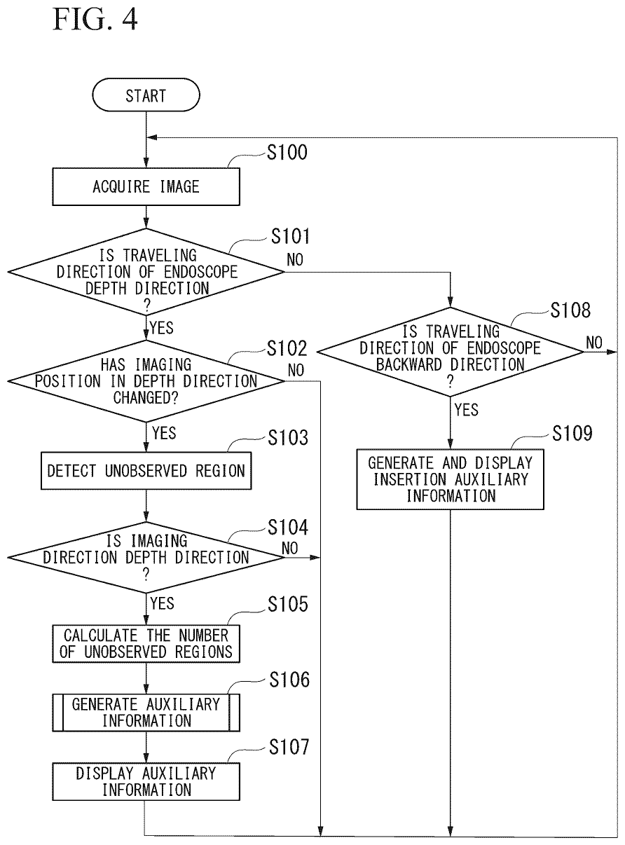

An example of an operation of the auxiliary information generation device 20 will be described using FIG. 4. FIG. 4 shows an example of a procedure of processing executed by the auxiliary information generation device 20.

### (Step S100)

The image acquisition unit 21 acquires an image output from the imaging unit 11. The image acquisition unit 21 outputs the image to the storage unit 22. The image output from the image acquisition unit 21 is output to the storage unit 22, and then output to the region detection unit 23, the imaging direction determination unit 25, and the display unit 27. The display unit 27 displays the image output from the storage unit 22.

### (Step S101)

The imaging direction determination unit 25 determines whether the traveling direction of the endoscope 10 is the depth direction based on the image output from the storage unit 22. When the traveling direction of the endoscope 10 is the depth direction, the imaging direction determination unit 25 outputs traveling direction information indicating the depth direction to the region detection unit 23. At this time, step S102 is executed. When the traveling direction of the endoscope 10 is not the depth direction, step S108 is executed.

### (Step S102)

The imaging direction determination unit 25 determines whether an imaging position in the depth direction has changed based on the image output from the storage unit 22. When the amount of change between an image of a current frame and an image of a frame immediately before the current frame is large, the imaging direction determination unit 25 determines that the imaging position in the depth direction has changed. When the amount of change is small, the imaging direction determination unit 25 determines that the imaging position in the depth direction has not changed. When the imaging position has changed, step S103 is executed. When the imaging position has not changed, step S100 is executed.

### (Step S103)

When the traveling direction information output from the imaging direction determination unit 25 indicates the depth direction, the region detection unit 23 detects an unobserved region in a section corresponding to a current imaging position based on the image output from the storage unit 22. The region detection unit 23 outputs region position information indicating the positions of the unobserved regions in the image to the region calculation unit 24.

For example, the region detection unit 23 performs camera position estimation and three-dimensional (3D) reconstruction by using a technology called visual simultaneous localization and mapping (SLAM). A 3D model is restored through 3D reconstruction. Positions on a 3D model and positions on an image are associated with each other.

When some regions of the digestive tract are not captured in the image or the image is not suitable for 3D reconstruction, some regions of the 3D model will not be restored. The region detection unit 23 detects these regions as unobserved regions.

The region detection unit 23 may process the image output from the storage unit 22 to detect a lesion. When a lesion is captured in an image generated when the imaging direction is the depth direction D1 shown in FIG. 3A and the lesion is not captured in an image generated when the imaging direction is the direction D2 shown in FIG. 3B, the region detection unit 23 may detect the lesion as an unobserved region. As described below, the region detection unit 23 may detect an unobserved region by using artificial intelligence (AI).

### (Step S104)

The imaging direction determination unit 25 determines whether the imaging direction of the imaging unit 11 is the depth direction based on the image output from the storage unit 22. When the imaging direction of the imaging unit 11 is the depth direction, the imaging direction determination unit 25 outputs imaging direction information indicating the depth direction to the region calculation unit 24 and the auxiliary information generation unit 26. At this time, step S105 is executed. When the imaging direction of the imaging unit 11 is not the depth direction, step S100 is executed.

### (Step S105)

When the imaging direction information output from the imaging direction determination unit 25 indicates the depth direction, the region calculation unit 24 calculates the number of unobserved regions. For example, the region calculation unit 24 calculates the number of unobserved regions in each of a plurality of image regions obtained by dividing the image output from the storage unit 22 in a clockwise direction.

For example, the image is divided into four image regions symmetrical around the center of the image. The four image regions are an upper right image region, an upper left image region, a lower left image region, and a lower right image region. The region calculation unit 24 calculates the number of unobserved regions in each image region based on the region position information output from the region detection unit 23. The region calculation unit 24 outputs the region number information indicating the number of unobserved regions in each image region to the auxiliary information generation unit 26.

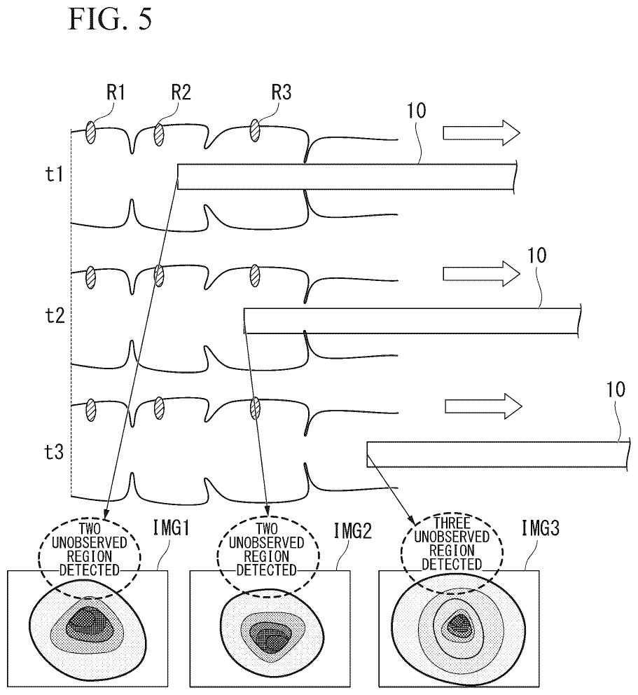

FIG. 5 shows an example of a method for calculating the number of unobserved regions. The endoscope 10 moves in a backward direction. The imaging unit 11 generates an image IMG1 at an imaging time t1, an image IMG2 at an imaging time t2, and an image IMG3 at an imaging time t3.

The region detection unit 23 detects unobserved regions R1 and R2 that are captured in the image IMG1 by using the image IMG1. The region detection unit 23 detects the unobserved regions R1 and R2 that are captured in the image IMG2 by using the image IMG2. The region detection unit 23 detects an unobserved region R3 that is captured in the image IMG3 by using the image IMG3. The unobserved regions R1 and R2 are not captured in the image IMG3. When the endoscope 10 passes through the same section in the digestive tract from the imaging time t1 to t3, the unobserved regions R1 to R3 are included in that section. For this reason, the number of unobserved regions in that section is three.

### (Step S106)

When the imaging direction information output from the imaging direction determination unit 25 indicates the depth direction, the auxiliary information generation unit 26 generates auxiliary information and outputs the auxiliary information to the display unit 27. Details of step S106 will be described below.

### (Step S107)

The display unit 27 displays the auxiliary information output from the auxiliary information generation unit 26. Examples of auxiliary information will be described below.

### (Step S108)

The imaging direction determination unit 25 determines whether the traveling direction of the endoscope 10 is a backward direction based on the image output from the storage unit 22. When the traveling direction of the endoscope 10 is the backward direction, the imaging direction determination unit 25 outputs traveling direction information indicating the backward direction to the region detection unit 23. At this time, step S109 is executed. When the traveling direction of the endoscope 10 is not the backward direction, step S100 is executed.

### (Step S109)

The auxiliary information generation unit 26 outputs insertion auxiliary information for assisting in insertion of the endoscope 10 to the display unit 27. The display unit 27 displays the insertion auxiliary information output from the auxiliary information generation unit 26.

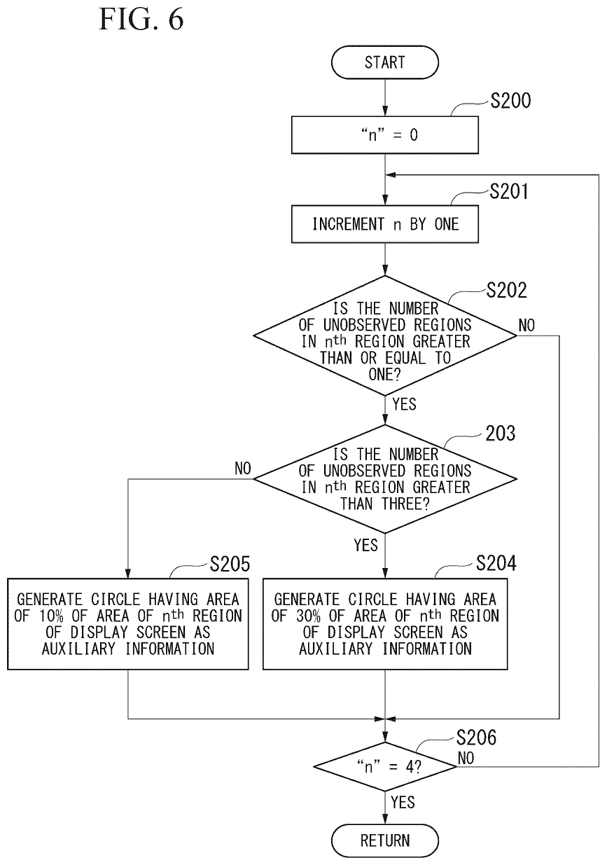

A first example of the operation of the auxiliary information generation device 20 in step S106 will be described using FIG. 6. FIG. 6 shows an example of a procedure of processing executed by the auxiliary information generation device 20 in step S106.

### (Step S200)

The auxiliary information generation unit 26 sets a variable n to 0, which is an initial value.

### (Step S201)

The auxiliary information generation unit 26 increments the variable n by 1.

### (Step S202)

The auxiliary information generation unit 26 refers to the region number information output from the region calculation unit 24 and determines whether the number of unobserved regions in an n^{th} region is greater than or equal to 1. A first region is an upper right image region. A second region is an upper left image region. A third region is a lower left image region. A fourth region is a lower right image region. When the number of unobserved regions in the n^{th} region is greater than or equal to 1, step S203 is executed. When the number of unobserved regions in the n^{th} region is 0, step S206 is executed.

### (Step S203)

The auxiliary information generation unit 26 determines whether the number of unobserved regions in the n^{th} region is greater than 3. When the number of unobserved regions in the n^{th} region is greater than 3, step S204 is executed. When the number of unobserved regions in the n^{th} region is equal to or less than 3, step S205 is executed. A threshold value of 3 in step S203 is an example. The threshold value is not limited to 3.

### (Step S204)

The auxiliary information generation unit 26 generates a circle having the area of 30% of the area of the n^{th} region of a display screen of the display unit 27 as auxiliary information.

### (Step S205)

The auxiliary information generation unit 26 generates a circle having the area of 10% of the area of the n^{th} region of the display screen of the display unit 27 as auxiliary information.

### (Step S206)

The auxiliary information generation unit 26 determines whether the variable n is 4. When the variable n is 4, step S107 shown in FIG. 4 is executed. When the variable n is not 4, step S201 is executed.

In steps S204 and S205, the auxiliary information generation unit 26 controls a size of an icon indicating auxiliary information of each image region according to the number of unobserved regions. When the number of unobserved regions is greater than or equal to a predetermined number, the auxiliary information generation unit 26 generates auxiliary information that is displayed in a first state. In the example described above, the first state is a circle having the area of 30% of the area of the n^{th} region of the display screen. When the number of unobserved regions is less than the predetermined number, the auxiliary information generation unit 26 generates auxiliary information that is displayed in a second state different from the first state. In the example described above, the second state is a circle with the area of 10% of the area of the n^{th} region of the display screen.

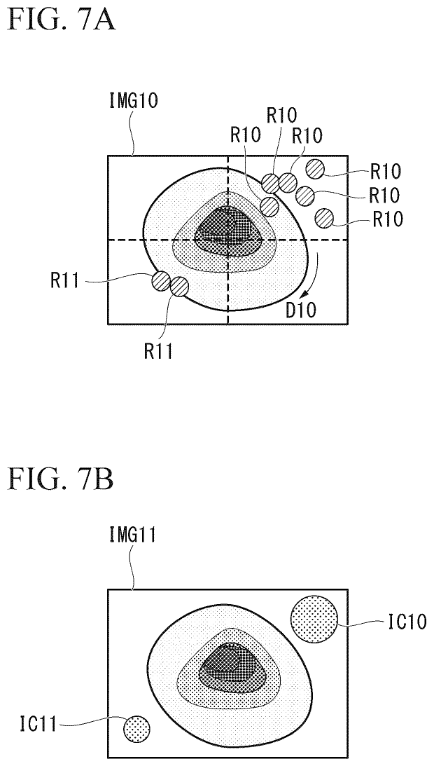

FIG. 7A shows an example of an unobserved region detected in an image. The region detection unit 23 detects six unobserved regions R10 in a first region of the image IMG10 output from the storage unit 22. The region detection unit 23 also detects two unobserved regions R11 in the third region of the image IMG10. The region calculation unit 24 calculates the number of unobserved regions in each of the plurality of image regions obtained by dividing the image IMG10 in the clockwise direction D10. The clockwise direction D10 is a circumferential direction of a circle whose center coincides with the center of the image IMG10.

In the example shown in FIG. 7A, the plurality of sections in the digestive tract correspond to four regions obtained by dividing the image IMG10 in the clockwise direction D10. When the imaging direction of the imaging unit 11 is the depth direction, the clockwise direction D10 corresponds to a circumferential direction of the digestive tract. The circumferential direction of the digestive tract is a clockwise direction in a cross section of the digestive tract perpendicular to the depth direction.

The user observes an image by placing emphasis on shading or a color of a relatively flat surface such as the inside of the digestive tract, a reproducibility of gradation expression of the image, or ease of understanding of the image, and observes a health condition or presence or absence of a lesion of an observation target so as not to miss even a minute change. If a large number of figures with contours are lined up, such as circles or frames indicating the unobserved regions R10 and R11, the user may find them irritating and complicated. Of course, even when they are displayed on a sub-screen different from a screen displaying an object, if miscellaneous information is lined up in a relatively small region of the sub-screen, an originally simple guide may become complicated and difficult to understand. If detailed figures are lined up in a small region, it becomes difficult for the user to quickly determine a meaning of their overlaps, and the like.

FIG. 7B shows an example of an image and auxiliary information displayed on the display unit 27. The display unit 27 displays an image IMG11. Icons IC10 and IC11 indicating auxiliary information are superimposed on the image IMG11.

An icon IC10 is a circle with an area that is 30% of the area of a first region of the image IMG11. The icon IC10 indicates the number of unobserved regions in the first region. The icon IC10 is superimposed on the first region. An icon IC11 is a circle with an area that is 10% of the area of a third region of the image IMG11. The icon IC11 indicates the number of unobserved regions in the third region. The icon IC11 is superimposed on the third region.

The icon IC10 and the icon IC11 are displayed as simplified circles, but may be displayed as polygons or arrows. Alternatively, frames indicating the icon IC10 and the icon IC11 may be displayed.

For example, if auxiliary information indicating the positions or the number of unobserved regions is displayed as many small icons or frames, the user will find them complicated. For this reason, the information described above is displayed as simplified and summarized icons or frames. As mentioned above, the user observes an image by placing emphasis on the shading or color of a relatively flat observation surface, the reproducibility of the gradation expression of the image, or the ease of understanding of the image, and observes the health condition or the presence or absence of a lesion of an observation target so as not to miss even a minute change. For such a user, to achieve the visibility of both an observed portion and auxiliary information (icons IC10 and IC11) in an image in which the observed portion is displayed, it may be preferable to simplify the auxiliary information. The auxiliary information generation device 20 switches the state of such auxiliary information according to the number of feature regions, thereby suppressing a sense of complicatedness or discomfort so that an image of an originally observed portion can be observed as much as possible.

The doctor can recognize presence of an unobserved region based on the icons IC10 and IC11. Since the icon IC10 is larger than the icon IC11, the doctor can determine that an unobserved region in an upper right region of a field of view of the endoscope 10 is greater than an unobserved region in a lower left region of the field of view of the endoscope 10.

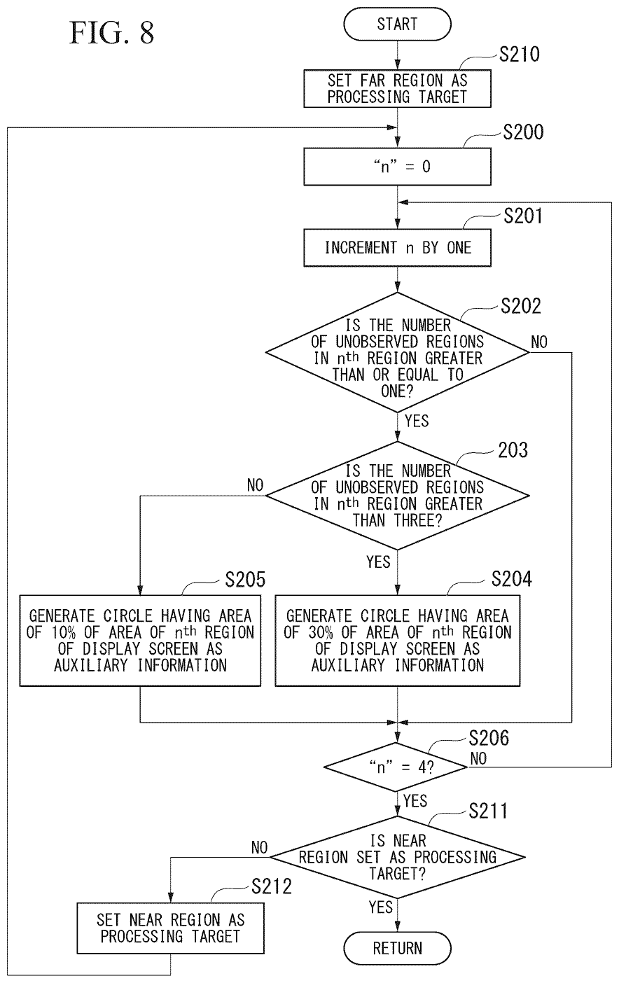

A second example of the operation of the auxiliary information generation device 20 in step S106 will be described using FIG. 8. FIG. 8 shows an example of the procedure of the processing executed by the auxiliary information generation device 20 in step S106. Processing that is the same as that shown in FIG. 6 will not be described. Processing that differs from the processing shown in FIG. 6 will be described.

### (Step S210)

The auxiliary information generation unit 26 sets a far region as a processing target. The far region is a region inside an ellipse that passes through a midpoint between the center of the image output from the storage unit 22 and the corner of the image. After step S210 is executed, step S200 is executed.

### (Step S211)

When the variable n is 4 in step S206, the auxiliary information generation unit 26 determines whether a near region is set as the processing target. The near region is a region outside the ellipse that passes through the midpoint between the center of the image output from the storage unit 22 and the corner of the image. In other words, the near region is a region outside the far region. When the near region is set as the processing target, step S107 shown in FIG. 4 is executed. When the far region is set as the processing target, step S212 is executed.

### (Step S212)

The auxiliary information generation unit 26 sets the near region as a processing target. After step S212 is executed, step S200 is executed.

In the example shown in FIG. 8, the image output from the storage unit 22 is divided into four image regions in the far region and four image regions in the near region.

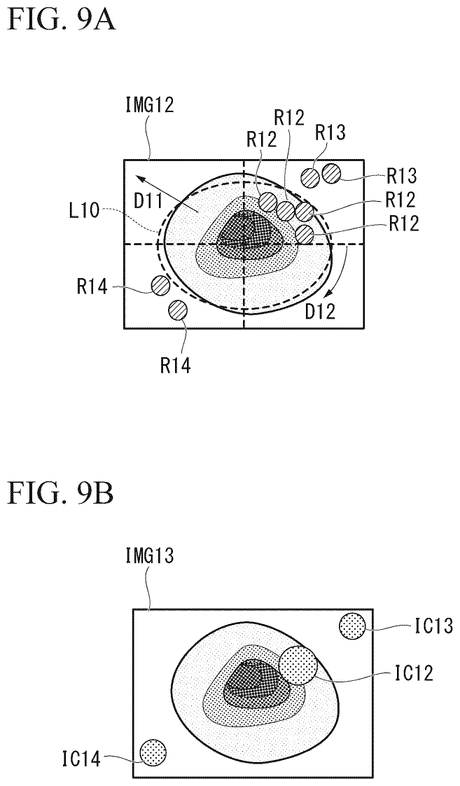

FIG. 9A shows an example of unobserved regions detected in an image. In an image IMG12 output from the storage unit 22, the far region is a region inside a line L10, and the near region is a region outside the line L10. The region detection unit 23 detects four unobserved regions R12 in a first region in the far region of the image IMG12. The region detection unit 23 also detects two unobserved regions R13 in a first region and two unobserved regions R14 in a third region in the near region of the image IMG12.

The region calculation unit 24 calculates the number of unobserved regions in each of the plurality of image regions obtained by dividing the image IMG10 output from the storage unit 22 in a direction D11 and a clockwise direction D12. The direction D11 is a direction from the center of the image IMG12 toward the outer periphery of the image IMG12. The clockwise direction D12 is a circumferential direction of a circle whose center coincides with the center of the image IMG12.

In the example shown in FIG. 9A, each section in the digestive tract corresponds to eight regions obtained by dividing the image IMG12 in the direction D11 and the clockwise direction D12. When the imaging direction of the imaging unit 11 is the depth direction, the direction D11 corresponds to a radial direction of the digestive tract, and the clockwise direction D12 corresponds to a circumferential direction of the digestive tract. The radial direction of the digestive tract is parallel to a line perpendicular to the depth direction that passes through the center of the cross section of the digestive tract perpendicular to the depth direction. The circumferential direction of the digestive tract is a clockwise direction on the cross section of the digestive tract perpendicular to the depth direction.

When a large number of figures with contours are lined up, such as circles or frames showing the unobserved regions R12 to R14, the user may find them irritating and complicated. Even when they are displayed on a sub-screen different from a screen displaying an object, if miscellaneous information is lined up in a relatively small region of the sub-screen, the originally simple guide may become complicated and difficult to understand.

FIG. 9B shows an example of the image and auxiliary information displayed on the display unit 27. The display unit 27 displays an image IMG13. Icons IC12, IC13, and IC14, which indicate auxiliary information, are superimposed on the image IMG13.

An icon IC12 is a circle with an area that is 30% of the area of a first region in a far region of the image IMG13. The icon IC12 indicates the number of unobserved regions in the first region in the far region. The icon IC12 is superimposed on the first region in the far region.

An icon IC13 is a circle with an area that is 10% of the area of a first region in a near region of the image IMG13. The icon IC13 indicates the number of unobserved regions in the first region in the near region. The icon IC13 is superimposed on the first region in the near region.

An icon IC14 is a circle with an area that is 10% of the area of a third region in the near region of the image IMG13. The icon IC14 indicates the number of unobserved regions in the third region in the near region. The icon IC14 is superimposed on the third region in the near region.

For example, if auxiliary information indicating the positions or the number of unobserved regions is displayed as many small icons or frames, the user will find it complicated. For this reason, the above information is displayed as simplified and summarized icons or frames. To achieve visibility of both an observed portion and auxiliary information (icons IC12 to IC14) in an image in which the observed portion is displayed, it may be preferable to simplify the auxiliary information. The auxiliary information generation device 20 reduces a sense of complicatedness or discomfort by switching the state of such auxiliary information according to the number of feature regions so that the image of the originally observed portion can be observed as much as possible.

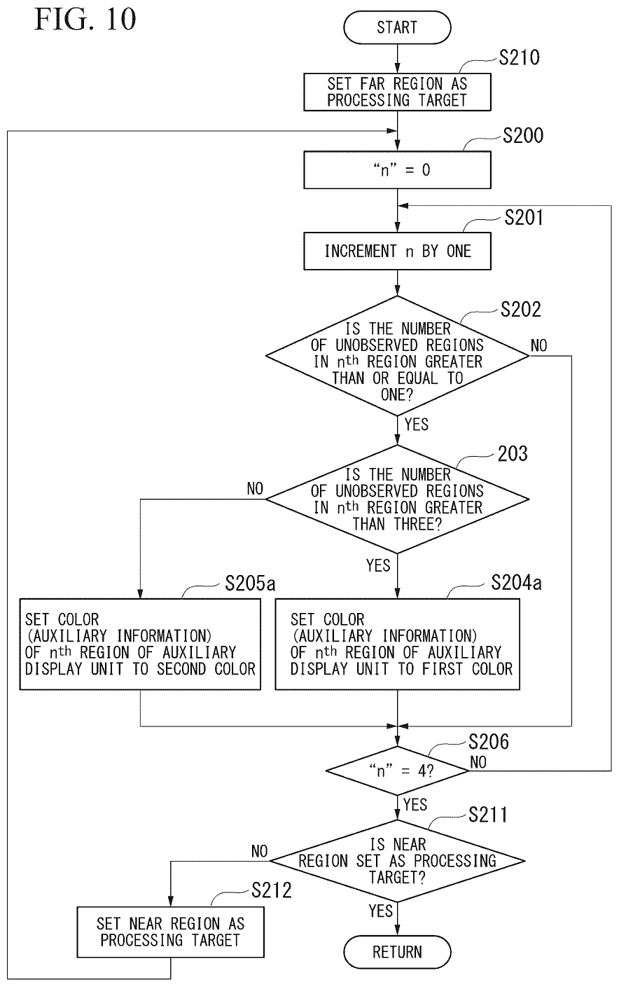

A third example of the operation of the auxiliary information generation device 20 in step S106 will be described using FIG. 10. FIG. 10 shows an example of the procedure of the processing executed by the auxiliary information generation device 20 in step S106. Processing that is the same as that shown in FIG. 8 will not be described. Processing that differs from the processing shown in FIG. 8 will be described.

Step S204 shown in FIG. 8 is changed to step S204a, and step S205 shown in FIG. 8 is changed to step S205a.

### (Step S204a)

The auxiliary information generation unit 26 sets a color of an n^{th} region of an auxiliary display unit to a first color. The auxiliary display unit is a part of the display screen of the display unit 27. The first color corresponds to the auxiliary information. For example, the first color is yellow, but is not limited to this.

### (Step S205a)

The auxiliary information generation unit 26 sets the color of the n^{th} region of the auxiliary display unit to a second color different from the first color. The second color corresponds to the auxiliary information. For example, the second color is red, but is not limited to this.

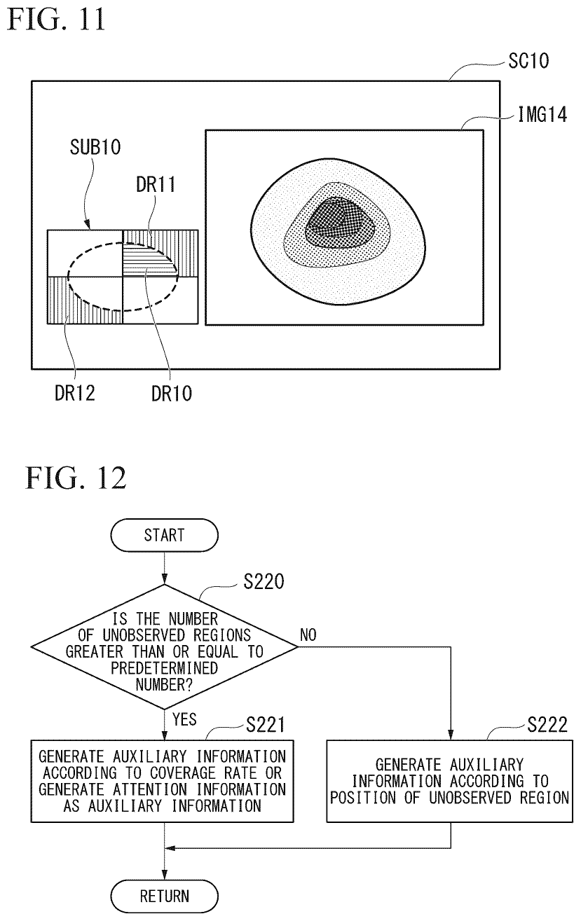

FIG. 11 shows an example of the display screen of the display unit 27. An image IMG14 is displayed on a display screen SC10. The display screen SC10 has an auxiliary display unit SUB10.

A display region DR10 of the auxiliary display unit SUB10 is displayed in a first color. The display region DR10 corresponds to the first region in the far region. The display regions DR11 and DR12 of the auxiliary display unit SUB10 are displayed in a second color. The display region DR11 corresponds to the first region in the near region, and the display region DR12 corresponds to the third region in the near region.

The region detection unit 23 sequentially acquires a plurality of images (image frames) obtained by the imaging unit 11 of the endoscope 10, and detects feature regions, for example, at a plurality of positions within the digestive tract of the subject, using the image frames or image frame groups. The region detection unit 23 may detect feature regions by using an inference model (AI) obtained by deep learning or the like using feature information appearing in the images as teacher data. The region detection unit 23 may also connect the plurality of images using 3D model construction technology, and determine that there is an unobserved region (a hole in a 3D model) when there are no image frames serving as a material for connecting the images. The region detection unit 23 may be realized by simply using a logic that determines feature information based on rules, image processing calculations, or the like.

In each embodiment of the present invention, the region detection unit 23 detects the feature regions described above by using an image or a group of images obtained in each of the plurality of sections in the digestive tract. The auxiliary information generation unit 26 determines the number of feature regions and generates auxiliary information according to the number of feature regions. The auxiliary information is displayed on the image in the display unit 27, or is displayed in a region (the display region DR11) different from a region in which the image is displayed in the display unit 27. When the auxiliary information is displayed in the display unit 27, the auxiliary information generation unit 26 controls a state of the auxiliary information according to the number of feature regions. For this reason, the auxiliary information generation unit 26 may need to use a result of recording a history of information detected by the region detection unit 23 as the number of feature regions depending on a situation. In this case, the region detection unit 23 may be equipped with a function for recording information.

The feature regions described above are assumed to be regions where oversight is likely to occur and regions where oversight has actually occurred (holes in the 3D model). Specific examples of regions where oversight is likely to occur are as follows:
First example: a region with residue, foam, or feces
Second example: a region with contracted mucosa
Third example: a region with bending lumen
Fourth example: a region with many folds or deep folds

As described above, the auxiliary information generation unit 26 generates auxiliary information according to the number of unobserved regions. When the auxiliary information is displayed on the display unit 27, the auxiliary information generation unit 26 controls the state of the auxiliary information according to the number of unobserved regions. For this reason, the auxiliary information generation unit 26 can avoid troublesome notification of auxiliary information and a decrease in visibility of the image.

### Second Embodiment

A second embodiment of the present invention will be described. In the second embodiment, the endoscope system 1 shown in FIG. 1 is used. The auxiliary information generation device 20 executes the processing shown in FIG. 4.

A first example of the operation of the auxiliary information generation device 20 in step S106 will be described using FIG. 12. FIG. 12 shows an example of the procedure of the processing executed by the auxiliary information generation device 20 in step S106.

### (Step S220)

The auxiliary information generation unit 26 refers to the region number information output from the region calculation unit 24 and determines whether the number of unobserved regions is greater than or equal to a predetermined number. When the number of unobserved regions is greater than or equal to the predetermined number, step S221 is executed. When the number of unobserved regions is less than the predetermined number, step S222 is executed.

### (Step S221)

The auxiliary information generation unit 26 generates auxiliary information according to a coverage rate. The coverage rate indicates a ratio of the area of a region of the digestive tract that does not include an unobserved region to the area of the entire region. Alternatively, the auxiliary information generation unit 26 generates an attention notice as the auxiliary information. The attention notice is an icon for notifying the user that there are the predetermined number of unobserved regions or more.

For example, the region detection unit 23 executes 3D reconstruction in step S103 shown in FIG. 4, and generates a 3D point group that constitutes a 3D model. The region detection unit 23 interpolates 3D point groups of a hole region in the 3D point groups. The hole region corresponds to an unobserved region. The auxiliary information generation unit 26 calculates a coverage rate CO (%) according to the following equation (1).

$$CO = 100 * ( 1 - N 1 / N 2 ) \qquad ( 1 )$$

N1 in Equation (1) indicates the number of interpolated 3D point groups. N2 in Equation (1) indicates the number of all 3D point groups including the interpolated 3D point groups.

The region detection unit 23 may generate mesh (polygon) data based on the 3D point groups. The region detection unit 23 may interpolate meshes of the hole region in a plurality of meshes. The auxiliary information generation unit 26 may calculate the coverage rate CO (%) according to the following equation (2) or equation (3).

$$CO = 100 * ( 1 - A 1 / A 2 ) \qquad ( 2 )$$

$$CO = 100 * ( 1 - N 3 / N 4 ) \qquad ( 3 )$$

A1 in Equation (2) indicates the area of an interpolated mesh, and A2 in Equation (2) indicates the area of the entire mesh including the interpolated mesh. N3 in Equation (3) indicates the number of interpolated meshes. N4 in Equation (3) indicates the number of the entire meshes including the interpolated meshes.

The auxiliary information generation unit 26 may generate auxiliary information according to the number of hole regions, instead of the coverage rate.

As described above, the user observes an image by placing emphasis on the shading or color of a relatively flat observation surface, the reproducibility of the gradation expression of the image, or the ease of understanding of the image, and observes the health condition or the presence or absence of a lesion of an observation target so as not to miss even a minute change. If a large number of figures with contours are lined up, such as the circles or frames indicating the unobserved region R10 and the unobserved region R11, the user may find them irritating and complicated.

### (Step S222)

The auxiliary information generation unit 26 generates auxiliary information corresponding to the positions of unobserved regions in the image.

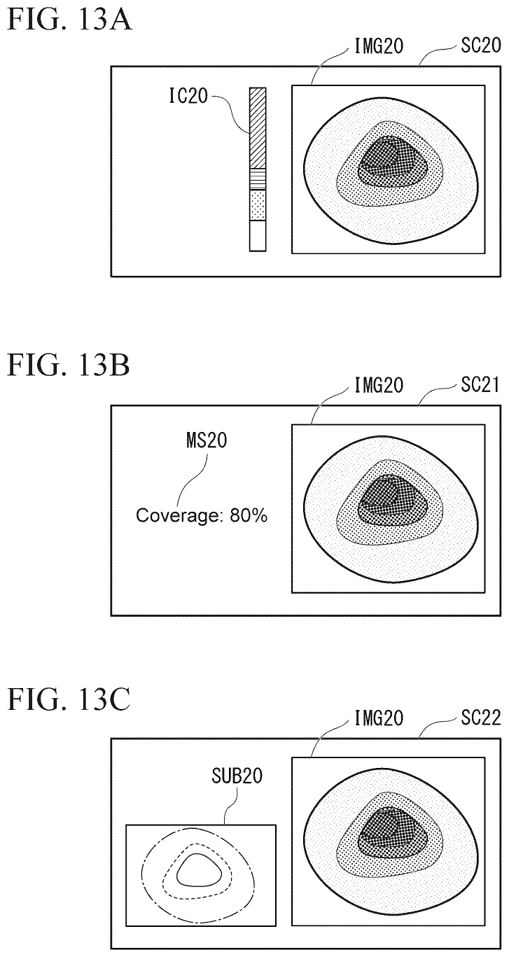

FIG. 13A shows a first example of the display screen of the display unit 27. An image IMG20 and an icon IC20 are displayed on a display screen SC20.

The icon IC20 constitutes the auxiliary information generated in step S221 and indicates the coverage rate of each of the plurality of sections in the digestive tract. The icon IC20 is constituted in a bar shape and is divided into a plurality of regions in a vertical direction. For example, a lower region in the icon IC20 indicates the coverage rate of a foreground section in the digestive tract captured in the image IMG20. An upper region in the icon IC20 indicates the coverage rate of a back section in the digestive tract shown in the image IMG20. Each region in the icon IC20 is displayed in a color according to the coverage rate of a section corresponding to that region.

FIG. 13B shows a second example of the display screen of the display unit 27. The image IMG20 and a message MS20 are displayed on a display screen SC21.

The message MS20 constitutes the auxiliary information generated in step S221 and indicates the coverage rate. The coverage rate in the message MS20 indicates the coverage rate of one section out of the plurality of sections in the digestive tract. The coverage rates of the plurality of sections may be displayed on the display screen SC21.

FIG. 13C shows a third example of the display screen of the display unit 27. The image IMG20 is displayed on a display screen SC22. The display screen SC22 has an auxiliary display unit SUB20.

The auxiliary display unit SUB20 displays a plurality of lines corresponding to the positions of the folds captured in image IMG20. The plurality of lines constitute auxiliary information generated in step S221. The plurality of sections in the digestive tract are separated by the folds. Each of the plurality of lines is displayed in a color corresponding to the coverage rate of each section.

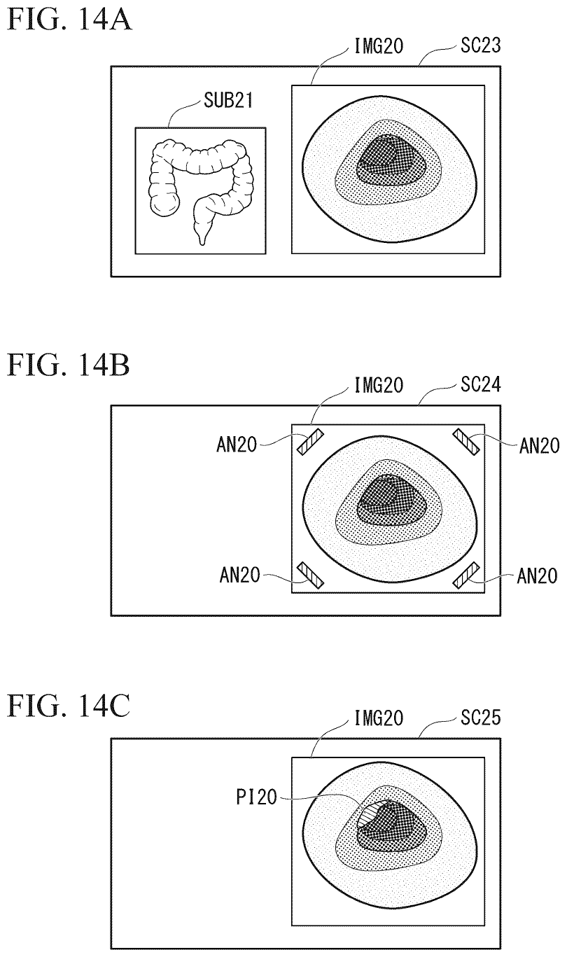

FIG. 14A shows a fourth example of the display screen of the display unit 27. The image IMG20 is displayed on a display screen SC23. The display screen SC23 has an auxiliary display unit SUB21.

The auxiliary display unit SUB21 displays a schematic diagram of the digestive tract that constitutes the auxiliary information generated in step S221. Each of the plurality of sections in the digestive tract corresponds to a portion such as a rectum. Each of the plurality of sections is displayed in a color that corresponds to the coverage rate of each section.

FIG. 14B shows a fifth example of the display screen of the display unit 27. The image IMG20 is displayed on a display screen SC24.

The attention notice AN20 is superimposed on the image IMG20. The attention notice AN20 indicates that there are a predetermined number of unobserved regions or more.

FIG. 14C shows a sixth example of the display screen of the display unit 27. The image IMG20 is displayed on a display screen SC25.

Position information PI20 is superimposed on the image IMG20. The position information PI20 constitutes auxiliary information generated in step S222, and indicates the position of an unobserved region. The position information PI20 is displayed at a position on the image IMG20 that corresponds to the position of an unobserved region. The position information PI20 may be displayed as an arrow indicating the position of an unobserved region.

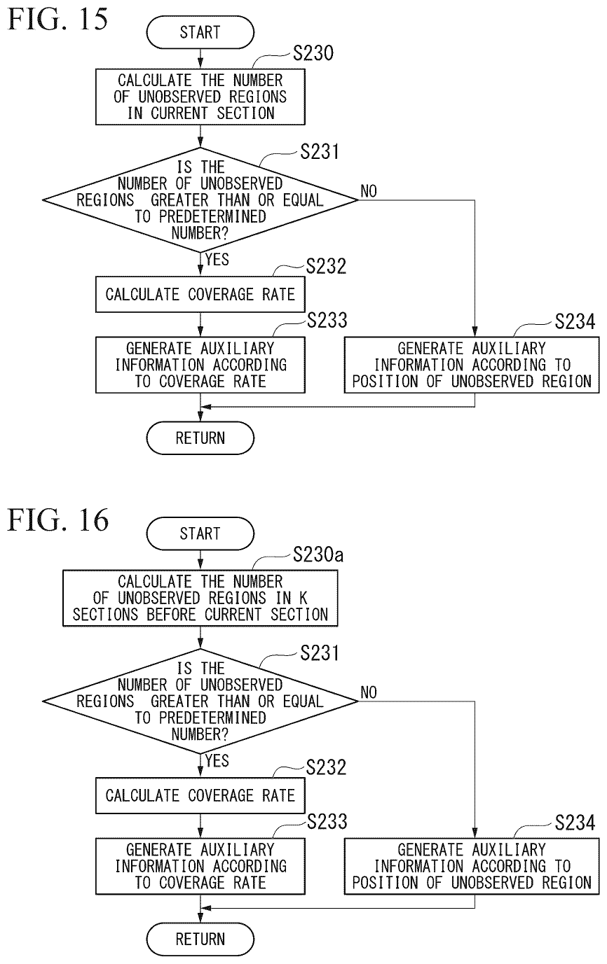

A second example of the operation of the auxiliary information generation device 20 in step S106 will be described using FIG. 15. FIG. 15 shows an example of the procedure of the processing executed by the auxiliary information generation device 20 in step S106.

### (Step S230)

Step S105 shown in FIG. 4 is included in step S230. The region calculation unit 24 calculates the number of unobserved regions in a section (current section) corresponding to a current imaging position. The number of unobserved regions in the current section does not include the number of unobserved regions in one or more sections (past sections) through which the endoscope 10 has already passed. The region calculation unit 24 outputs region number information indicating the number of unobserved regions to the auxiliary information generation unit 26.

### (Step S231)

The auxiliary information generation unit 26 refers to the region number information output from the region calculation unit 24 and determines whether the number of unobserved regions is greater than or equal to a predetermined number. When the number of unobserved regions is greater than or equal to the predetermined number, step S232 is executed. When the number of unobserved regions is less than the predetermined number, step S234 is executed.

### (Step S232)

The auxiliary information generation unit 26 calculates a coverage rate according to the number of unobserved regions. A method of calculating the coverage rate is the same as that of calculating the coverage rate in step S221 shown in FIG. 12.

### (Step S233)

The auxiliary information generation unit 26 generates auxiliary information according to the coverage rate.

### (Step S234)

The auxiliary information generation unit 26 generates auxiliary information indicating the positions of the unobserved regions in the image.

Using FIG. 16, a third example of the operation of the auxiliary information generation device 20 in step S106 will be described. FIG. 16 shows an example of the procedure of the processing executed by the auxiliary information generation device 20 in step S106. Processing that is the same as that shown in FIG. 15 will not be described. Processing that differs from the processing shown in FIG. 15 will be described.

Step S230 shown in FIG. 15 is changed to step S230a.

### (Step S230a)

The region calculation unit 24 calculates the number of unobserved regions in one or more sections (past sections) through which the endoscope 10 has already passed. The number of unobserved regions in the past sections does not include the number of unobserved regions in a section (current section) corresponding to a current imaging position. The region calculation unit 24 outputs region number information indicating the number of unobserved regions to the auxiliary information generation unit 26.

Once steps S232 and S233 are executed, execution of steps S231 and S234 may be stopped. In other words, after step S230a is executed, step S232 may be executed without executing step S231.

As described above, the auxiliary information generation unit 26 generates auxiliary information according to the number of unobserved regions. When the number of unobserved regions is large, the auxiliary information generation unit 26 generates auxiliary information indicating the coverage rate. The user can roughly grasp presence of the unobserved regions. When the number of unobserved regions is small, the auxiliary information generation unit 26 generates auxiliary information indicating the positions of the unobserved regions. The user can grasp detailed positions of the unobserved regions.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. An auxiliary information generation method comprising:
sequentially acquiring a plurality of images obtained by an image sensor of an endoscope at a plurality of positions in a digestive tract of a subject;
detecting feature regions in the digestive tract based on the plurality of images;
calculating a number of the feature regions in each of a plurality of sections in the digestive tract;
generating auxiliary information according to the number of the feature regions;
determining whether the image sensor is executing imaging in a depth direction of the digestive tract; and
controlling a state of the auxiliary information according to the number of the feature regions when it is determined that the image sensor is executing imaging in the depth direction and the auxiliary information is displayed on a display.

2. The auxiliary information generation method according to claim 1, further comprising:
generating the auxiliary information for each of a plurality of image regions obtained by dividing an image obtained by the image sensor in a direction corresponding to a circumferential direction of the digestive tract.

3. The auxiliary information generation method according to claim 2, further comprising:
controlling a size of an icon indicating the auxiliary information for each of the plurality of image regions according to the number of the feature regions.

4. The auxiliary information generation method according to claim 1, further comprising:
dividing an image obtained by the image sensor in a direction corresponding to a radial direction of the digestive tract; and
generating the auxiliary information for each of a plurality of image regions obtained by dividing the image in a direction corresponding to a circumferential direction of the digestive tract.

5. The auxiliary information generation method according to claim 1, further comprising:
generating the auxiliary information displayed in a first state when the number of the feature regions is greater than or equal to a predetermined number; and
generating the auxiliary information displayed in a second state different from the first state when the number of the feature regions is less than the predetermined number.

6. The auxiliary information generation method according to claim 5,
wherein the auxiliary information displayed in the second state indicates positions of the feature regions.

7. An auxiliary information generation device comprising a processor configured to:
detect feature regions at respective positions along a lumen within the lumen based on an image obtained by an image sensor at a distal end of an endoscope to calculate a number of feature regions at the respective positions;
determine an imaging direction of the image sensor within the lumen; and
generate auxiliary information according to the number of the feature regions at the respective positions when the imaging direction is determined to be a depth direction of the lumen and the auxiliary information is displayed on a display.

8. A computer-readable non-transitory storage medium that has recorded a program causing a computer to execute:
detecting feature regions at respective positions along a lumen within the lumen based on an image obtained by an image sensor at a distal end of an endoscope to calculate a number of feature regions at the respective positions;
determining an imaging direction of the image sensor within the lumen; and
generating auxiliary information according to the number of the feature regions at the respective positions when the imaging direction is determined to be a depth direction of the lumen and the auxiliary information is displayed on a display.
